(19) United States
(12) Patent Application Publication
(10) Pub. No.: US 2012/0220544 A1
(43) Pub. Date: Aug. 30, 2012

(54) Ganglioside Transmucosal Formulations

(76) Inventors: Jay S. Schneider, Cherry Hill, NJ (US); John J. Koleng, Austin, TX (US); Robert Florentine, Naples, FL (US); David W. Anderson, West Chester, PA (US)

(21) Appl. No.: 13/407,296

(22) Filed: Feb. 28, 2012

Related U.S. Application Data
(63) Continuation of application No. PCT/US10/47527, filed on Sep. 1, 2010.
(60) Provisional application No. 61/238,748, filed on Sep. 1, 2009.

(51) Int. Cl. A61K 31/7028; A61M 31/00; A61M 11/00; A61P 25/00; A61P 25/16

(52) U.S. Cl. 514/25; 128/200.14; 604/286

(57) ABSTRACT

A transmucosal formulation comprising a ganglioside and a mucosal absorption enhancer, as well as a method of treating or preventing Parkinson's disease in a human patient in need thereof comprising parenterally administering such a transmucosal formulation to said patient.

### RELATED APPLICATIONS

This application is a continuation of International Application No. PCT/US10/47527, which designated the United States and was filed on Sep. 1, 2010, published in English, which claims the benefit of U.S. Provisional Application No. 61/238,748 filed Sep. 1, 2009, herein incorporated by reference in its entirety.

### CROSS REFERENCE TO RELATED APPLICATIONS

This application is also related to PCT Application Nos. PCT/US10/47522; PCT/US10/47524; PCT/US10/47528 and PCT/US10/47520, all filed Sep. 1, 2010 and based on US Provisional Application Nos.: 61/238,775; 61/238,726; 61/238,735 and 61/238,712, each of which is incorporated by reference in its entirety.

### FIELD

Disclosed herein are transmucosal formulations of gangliosides, e.g., monosialoganglioside (GM1), and their use in their treatment of neurodegenerative disease, and more particularly for therapy of Parkinson's Disease.

### BACKGROUND

Parkinson's disease (PD) is a slowly but relentlessly progressive, neurodegenerative disorder resulting in a time-dependent worsening of clinical symptoms. Clinical symptoms include tremor, bradykinesia (slowed motion), rigid muscles, impaired posture and balance, loss of automatic movements, and speech changes. Although there is considerable clinical variability between patients, the current armamentarium of anti-PD drugs effectively, if albeit temporarily, ameliorates most of the major parkinsonian signs and symptoms in a majority of patients. Despite transient symptomatic improvements from traditional drug therapies, functional disability worsens over time.

The advent of levodopa therapy has been associated with a prolongation of survival in PD patients but is not associated with slowing the progression of symptoms. Levodopa, a metabolic pre-cursor of dopamine (L-3,4-dihydroxy phenylalanine), presently is the single most effective agent in the treatment of PD. Administered in connection with levodopa to prevent the catabolization of levodopa administered orally are catechol-O-methyltransferase (COMT) inhibitors such as tolcapone and entacapone; therefore, increasing the plasma half-life and the percentage of levodopa that reaches the Central Nervous System (CNS). A continuing problem with levodopa therapy is that after a long efficacy period in patients, the effectiveness in reducing symptoms last shorter after each dose. Additionally, dyskinesia occurs over time. These effects of continued use of levodopa are a result of progressive dopamine degeneration. No drug has yet been identified that definitively slows or stops the progression of PD or substantially forestalls the inevitable functional decline in PD patients.

Drugs that can modify clinical progression, remediate motor and/or cognitive deficits, restore or enhance function of residual parts of the dopamine (“DA”) system or activate compensatory mechanisms are sorely needed. No agent studied to date, however, has yielded convincing evidence of neuroprotection or disease modification and no agent has been studied as a neurorestorative agent.

Preclinical in vitro and in vivo studies have shown GM1 to rescue damaged DA neurons, stimulate survival and repair of dopaminergic neurons and stimulate sprouting of functional dopaminergic terminals, increase DA levels in the striatum and up-regulate DA synthetic capacity of residual nigrostriatal neurons. See, e.g., “GM1 Ganglioside in the Treatment of Parkinson's Disease,” Schneider, Ann. N.Y. Acad. Sciences 845, 363-73 (February 2006). Preliminary clinical studies of GM1 in PD patients also showed clinical improvements in patients with short-term use of GM1 and minimal symptom progression in a sub-group of patients followed over five years of GM1 use, followed by significant progression of symptoms following discontinuation of long-term GM1 use. Therefore, a potentially fruitful approach to the treatment of PD consists of administration of agents such as GM1, which may stabilize injured or dying DA neurons, stimulate sprouting of new dopaminergic fibers and terminals, and/or enhance the function of residual dopaminergic neurons or stimulate or maintain compensatory processes.

GM1, a monosialoganglioside, is a normal constituent of nerve cell membranes, and is known to modulate a number of cell surface and receptor activities as well as play important roles in neuronal differentiation and development, protein phosphorylation, and synaptic function. In numerous preclinical studies, chronic treatment with GM1 following different types of lesions to the central nervous system has resulted in biochemical and behavioral recovery and these effects have been particularly impressive in the damaged DA system.

Heretofore, GM1 has been administered by parenteral injection due to breakdown of GM1 in the gastrointestinal system. This route of administration, however, is difficult for patients who must self-administer the injection. This disadvantage is especially significant for Parkinson's patients who have significant problems with tremor and cannot easily self-administer an injection. This route of administration is also inefficient and requires a large amount of GM1 to be injected in order to deliver a therapeutic amount of GM1 to the brain, the intended site of benefit.

A continuing and unmet need exists for new and improved parenteral formulations of GM1, particularly formulations that can be easily self-administered by Parkinson's patients and that can reduce the amount of GM1 required to be administered to achieve a therapeutic response.

### SUMMARY

There are many other neurodegenerative diseases (e.g., progressive supranuclear palsy, a variety of different “Parkinson's-Plus” syndromes, amyotrophic lateral sclerosis, Alzheimer's disease, spinal muscular atrophy, multisystem atrophy, Friedreich's ataxia, olivopontocerebellar atrophy) that and may be amenable to GM1 therapy. There is also the potential utility of this therapy to treat a variety of acquired brain injuries such as traumatic brain injury, spinal cord injury, coronary bypass graft surgery-induced cognitive impairment, and chemotherapy or radiation therapy induced cognitive disorder. This application describes parenteral transmucosal formulations of gangliosides, e.g., the monosialoganglioside GM1, and their use in the treatment or prevention of Parkinson's disease.

Described is a new parenteral transmucosal formulations of gangliosides, in particular, the monosialoganglioside GM1, and their use in the treatment or prevention of Parkinson's disease.

An aspect of the invention provides for a transmucosal formulation comprising a ganglioside and a mucosal permeation enhancing agent.

In another aspect, disclosed in this invention is a pharmaceutical composition for treatment or prevention of a CNS disease or condition in a patient amenable to treatment by therapeutic administration of an GM1, that comprises a liquid, gel, or powder formulation for transmucosal administration with GM1 and at least one permeation-enhancing agent effective to enhance transmucosal drug uptake; at least one buffer; at least one solvent; and at least one osmolarity agent.

Additional features may be understood by referring to the following detailed description and examples.

### DETAILED DESCRIPTION

Described herein are new transmucosal formulations comprising a ganglioside GM1 and a mucosal absorption enhancer, as well as methods of treating or preventing Parkinson's disease in a human patient in need thereof comprising parenterally (e.g., buccally or intranasally) administering such a transmucosal formulation to the patient.

It is an embodiment of the invention to provide for a transmucosal formulation comprising a therapeutically effective amount of a ganglioside, specifically GM1 and a mucosal permeation enhancing agent. Also an embodiment, is a method of treating or preventing a neuromuscular disease in a human patient in need by parenterally administering a transmucosal formulation comprising a therapeutically effective amount to a patient.

Another embodiment of the invention discloses a pharmaceutical composition for treatment or prevention of a CNS disease or condition in a patient amenable to treatment by therapeutic administration of an GM1, comprising a liquid, gel, or powder formulation for transmucosal administration comprising: GM1 and at least one permeation-enhancing agent effective to enhance transmucosal drug uptake; at least one buffer; at least one solvent; and at least one osmolarity agent, wherein the pharmaceutical corporation treats the CNS disease or condition of Parkinson's disease. The liquid or gel solution can be an aqueous solution, pr where the solution can be a solution in a liquid for transmucosal administration either buccally/orally or intranasally.

In one embodiment, the permeation-enhancing agent can be selected from the group consisting of: alkyl glycosides, tetra-decyl maltoside (TDM), lysophosphatidylcholine, sodium glycochoate, didecanoylphosphatidylcholine (DDPC), cyclodextrins, lauroylcarnitine chloride (LLC), aminated gelatin, SLS and any combination thereof. The GM1 is either naturally or synthetically derived. The solvent can be water and the osmolarity agent is selected from the group consisting of sodium chloride, dextrose, sorbitol.

In one embodiment, the GM1 can be either synthetically or naturally derived or isolated from cultures of GM1 producing cells isolated from mammals, especially non-bovine mammals, as are known in the art. Such methods can be found in U.S. patent application Ser. No. ______, herein incorporated by reference in its entirety.

In another embodiment, the pharmaceutical composition is a co-solvent selected from the group selected from: propylene glycol, polyethylene glycol, ethanol and any combination thereof, and a viscosity agent which is a polymer selected from the group consisting of MC, HPMC, PVP, HEC, NaCMC, microcrystalline cellulose, Hydroxypropyl Cellulose, hydroxyethyl cellulose, polyvinylpyrrolidone and any combination thereof. The composition may also have a pH between about 7.2 to about 8.2 and more particularly, 7.4.

In another embodiment, the pharmaceutical composition also comprises a chelating agent such as sodium EDTA or disodium EDTA dehydrate, and a preservative selected from the group consisting of phenylethyl alcohol, potassium sorbate, benzyl alcohol. The pharmaceutical composition may comprise a co-therapeutic. In one embodiment, the pharmaceutical composition is substantially free of BSE contaminants. The composition disclosed herein is administered transdermally in an amount less than 200 mg, which is typically administered subcutaneously. The GM1 of the composition can be administered to a patient in an effective dose of between about 0.1 mg and about 100 mg. The GM1 may be administered to the patient in an effective dose between 0.1 mg to up and about 200 mg. The composition may be administered in a therapeutically effective amount. The pharmaceutical composition may have a membrane stabilizing agent to reduce nasal irritation.

In yet another embodiment, the permeability enhancing agent is a mucoadhesive agent, wherein nasal resident time and nasal absorption is increased enhancing retention time of the composition and bioavailability of GM1. The mucoadhesive can be a chitosan, a chitosan derivative or a mucoadhesive polymer. The permeability enhancing agent comprises a tri-block co-polymer wherein nasal resident time and nasal absorption can be increased enhancing retention time of the composition and bioavailability of GM1. The permeability enhancing agent can also be a mucoadhesive agent, wherein oral/buccal resident time and buccal absorption can increase enhancing retention time of the composition and bioavailability of GM1.

In another embodiment, the pharmaceutical composition following intranasal administration to said patient yields a peak concentration of said GM1 in a central nervous system tissue or fluid of said patient in a biologically relevant amount. The transmucosal administration involves delivery of said composition to one or both nasal mucosal surfaces of said patient.

Another aspect of the invention discusses a method for treating or preventing a disease or condition in a patient in need of treatment by therapeutic administration of GM1 comprising the step of administering intranasally a pharmaceutical composition to a patient and is administered as a single solution in a multidose or single dose nasal dispenser. The administration may involve delivery of pharmaceutical composition to a nasal mucosal surface of said patient. GM1 is administered to a patient in an effective dose of between about 0.1 mg and 100 mg, or GM1 is administered to said patient in an effective dose of 0.1 mg to up to about 200 mg that yields a peak concentration of said GM1 in a central nervous system tissue or fluid of said patient in a biologically relevant amount that is at least 15% of the peak concentration of said GM1 or at least 20% of the peak concentration of said GM1 in a blood plasma of said patient, or that is at least 40% of the peak concentration of said GM1 in a blood plasma of said patient.

In another aspect, the pharmaceutical composition following mucosal administration to said patient yields a peak concentration of said GM1 in a central nervous system tissue or fluid of said subject that is greater than a therapeutic concentration of said GM1 in the plasma of said subject. In yet another aspect, the pharmaceutical composition following transmucosal administration to said patient yields an increase in central nervous system tissue or fluid concentration of GM1 in comparison to patients following subcutaneous administration of an equivalent amount of said GM1.

Another aspect of the invention provides an article of manufacture as a means for administering a nasal dose along with the composition where the means for administering a nasal dose is a nasal dispenser, tampon, sponge, insufflator, nebulizer or pump and in a package suitable for sale and distribution.

In certain embodiments the permeation-enhancing agent of the formulation may be selected from: an aggregation inhibitory agent; a charge modifying agent; a pH control or buffering agent; a redox control or buffering agent a degradative enzyme inhibitory agent; a mucolytic or mucus clearing agent; a ciliostatic agent; an absorption enhancement agent selected from a surfactant, a bile salt, a phospholipid additive, mixed micelle, liposome, or carrier, an alcohol, an enamine, an NO donor compound, a long-chain amphipathic molecule; a small hydrophobic penetration enhancer; sodium or a salicylic acid derivative; a glycerol ester of acetoacetic acid a cyclodextrin or β-cyclodextrin derivative, a medium-chain fatty acid, a chelating agent, an amino acid or salt thereof, an N-acetylamino acid or salt thereof, an enzyme degradative to a selected membrane component, an inhibitor of fatty acid synthesis, or an inhibitor of cholesterol synthesis; or any combination of the membrane penetration enhancing agents; a modulatory agent of epithelial junction physiology; a vasodilator agent; a stabilizing delivery vehicle, carrier, support or complex-forming species with which the GM1 can be effectively combined, associated, contained, encapsulated or bound resulting in stabilization of the GM1 for enhanced transmucosal delivery, wherein the formulation of the GM1 with the one or more delivery-enhancing agents provides for increased bioavailability of GM1 in a central nervous system tissue or fluid of the subject; a humectant or membrane stabilizing agent; and a permeation-enhancing peptide agent or any combination thereof.

A problem associated with the current therapeutic regimens for the chronic use of GM1 in PD patients is the necessity to deliver the drug by subcutaneous injection. Dislike of injections or inability to self-administer by this route makes subcutaneous treatment difficult for many PD patients. This invention overcomes this problem by using new formulations of GM1 together with absorption enhancers or mucoadhesive polymers in a preparation that can be administered intranasally for direct access to brain, bypassing the blood brain barrier, or by oral mucosal absorption, thus bypassing first pass metabolism in the gut/liver. The oral mucosa, target of buccal/oral administration methods includes all mucous membrane epithelium of the mouth, oro-pharynx and throat, the oral cavity, glands, tongue, vestibule, lip, cheek (buccal pad) gingival and palate. For preferred oral embodiment, buccal administration sublingual delivery is preferred.

Accordingly, GM1 ganglioside, either naturally derived from porcine, bovine or ovine brain or synthetically manufactured, is used alone or together with other gangliosides in a preparation with mucosal absorption enhancers and or mucoadhesive polymers for intranasal or oral mucosal administration to, for example, Parkinson's disease patients and potentially patients with other types of neurodegenerative disorders as a neuroprotective or neurorestorative drug for cognitive and motor dysfunction. In addition to Parkinson's disease therapy as disclosed herein the composition and method may be used to treat a variety of neurodegenerative disorders including, without limitation: any disease amenable to treatment by administration of GM1; Parkinson's-like dementia, Huntington's Disease; Huntington's-type dementia; and Alzheimer's disease.

Gangliosides can be administered alone or together with standard medical care for PD patients (or patients with other types of neurodegenerative diseases). Methods for producing ganglioside include those set forth in U.S. patent application No. ______, herein incorporated by reference in its entirety. U.S. Pat. No. ______ and U.S. Pat. No. ______.

Suitable parenteral dosage forms include GM1 in combination with at least one mucosal absorption enhancer, optionally with other gangliosides. Such dosage forms may be administered transmucosally, e.g., via nasal or mucosal administration. Dosage forms may also include prolonged action dosage forms or controlled release formulations (liposomes, nanoparticles, microspheres) to prolong drug activity. Still other dosage forms include gangliosides coupled to appropriate transporter molecules in order to cross the blood brain barrier following intranasal or mucosal administration.

Formulations for intranasal administration may include a therapeutic dose of GM1 together with permeation enhancing agents to enhance movement across the nasal membranes and to gain entrance to the brain, i.e., permeation enhancers, including but not limited to alkyl glycosides (e.g., tetra-decyl maltoside (TDM)), lysophosphatidylcholine, sodium glycochoate, didecanoylphosphatidylcholine (DDPC), cyclodextrins, lauroylcarnitine chloride (LLC), and aminated gelatin.

In one embodiment, formulations comprise a dosing regimen of a target dose of about 20 mg/day GM-1 wherein the dose deposition is on olfactory epithelium via 1 Spray/Nostril BID (5 mg/Spray) formulation. Target drug concentration can be about 5 mg/100 μL (i.e. 5% w/v). These aqueous-based compositions can be delivered via pressurized (i.e. propellant gas) or non-pressurized delivery (i.e., mechanical pumps).

TABLE 1

| Function | Requirement | % Range | Examples |
|---|---|---|---|
| GM-1 | Yes | 5 | GM-1 |
| Vehicle/Solvent | Yes | 75-95 | Purified Water |
| Osmolality | Yes | 0.5-5 | Sodium Chloride, |
| | | | Dextrose, Sorbitol |
| CoSolvent | Optional | 0-20 | Propylene Glycol, PEG, |
| | | | Ethanol |
| Chelating Agent | Optional | 0-0.25 | Sodium EDTA |
| Preservative | Optional | 0-2 | Phenylethyl Alcohol, |
| | | | Potassium Sorbate, Benzyl |
| | | | Alcohol |
| Permeation | Maybe | 0-5 | TDM, Cyclodextrin, SLS, |
| Enhancer | | | |
| Viscosity | Optional | 0-1 | MC, HPMC, PVP, HEC, |
| | | | NaCMC |
| Miscellaneous | Optional | 0-1 | Antioxidants, Fragrance, |
| | | | Flavors, Etc. |

TABLE 2

| | | Formulation (mg/100ul) | | | |
|---|---|---|---|---|---|
| | Func- | | | | MCG1- |
| Component | tion | MCG₁₋₁₋₁ | MCG₁₋₁₋₂ | MCG₁₋₂₋₁ | 2-2 |
| GM-1 | Drug | 5.00 | 5.00 | 5.00 | 5.00 |
| | Sub- | | | | |
| | stance | | | | |
| Polymer | Vis- | Avicel | Klucel HF | Natrosol | Kollidon |
| | cosity | RC-591 | 0.25 | 250H | 90F |
| | | 1.0 | | 0.25 | 1.00 |
| NaPhosphate, | Buffer | 0.12 | 0.12 | 0.12 | 0.12 |
| Monobasic, | | | | | |
| Monohydrate | | | | | |
| NaPhosphate, | Buffer | 0.03 | 0.03 | 0.03 | 0.03 |
| Dibasic, | | | | | |
| Dihydrate | | | | | |
| TDM | Pene- | 0.20 | 0.20 | — | — |
| | tration | | | | |
| | En- | | | | |
| | hancer | | | | |
| Benzalkonium | Preser- | 0.02 | 0.02 | 0.02 | 0.02 |
| Chloride (50%) | vative | | | | |
| Disodium | Che- | 0.10 | 0.10 | 0.10 | 0.10 |
| EDTA | lating | | | | |
| Dihydrate | Agent | | | | |
| Purified Water | Vehicle/ | Qₛ | Qₛ | Qₛ | Qₛ |
| | Solvent | | | | |

TABLE 3

| | | Formulation (mg/100ul) | | | |
|---|---|---|---|---|---|
| Component | Function | MCG₁₋₄₋₁ | MCG₁₋₄₋₂ | MCG₁₋₅₋₁ | MCG₁₋₅₋₂ |
| GM-1 | Drug | 5.00 | 5.00 | 5.00 | 5.00 |
| | Susbstance | | | | |
| Polymer | Viscosity | — | — | Methocel A₄M | Methocel E₄M |
| | | | | 0.10 | 0.25 |
| NaPhosphate, | Buffer | 0.24 | — | 0.24 | 0.24 |
| Monobasic, | | | | | |
| Monohydrate | | | | | |
| NaPhosphate, | Buffer | 0.06 | — | 0.06 | 0.06 |
| Dibasic, | | | | | |
| Dihydrate | | | | | |
| Anhydrous | Buffer | — | 0.02 | — | — |
| Citric Acid | | | | | |
| Sodium Citrate | Buffer | — | 0.23 | — | — |
| Anhydrous | | | | | |
| Sodium | Osmolarity | 0.50 | 0.50 | 0.50 | 0.50 |
| Chloride | | | | | |
| Phenylethyl | Preservative | 0.25 | 0.25 | 0.25 | 0.25 |
| Alcohol | | | | | |
| Disodium | Chelating Agent | 0.10 | 0.10 | 0.10 | 0.10 |
| EDTA | | | | | |
| Dihydrate | | | | | |
| Purified Water | Vehicle/Solvent | Qₛ | Qₛ | Qₛ | Qₛ |

Suitable formulations for intranasal administration may also include a therapeutic dose of GM1 in a gel formulation with in situ gelling and mucoadhesive properties such that there is increased permeation and prolonged nasal residence time and thereby increased nasal absorption. Such formulations would increase retention time of the GM1 in the nasal cavity resulting in greater bioavailability and greater transfer of GM1 to the brain via the olfactory pathway. As an example, chitosan-based mucoadhesive formulations may be used to enhance the retention time and bioavailability of GM1. Nasal bioadhesive gels could also provide enhanced bioavailability compared with other delivery routes and be combined in a formulation with other absorption enhancers.

Such a formulation may include a therapeutically active amount of GM1 together with gelling solutions of tri-block copolymers of poly(ethylene oxide) and polypropylene oxide) (e.g., Pluronic F127 (“PF127”)) that exhibit thermoreversible properties. By modulating the gelation temperature of different PF 127 solutions, liquid bases for nasal use can be formulated that form a gel in the nasal cavity at body temperature with suitable gel strength resulting in enhancement of the residence time in the nasal cavity. The high solubilizing capacity and nontoxic properties of PF127 make it suitable for nasal drug delivery. GM1 formulations for intranasal delivery may therefore include thermoreversible polymer PF 127 and a mucoadhesive polymer (such as C934P), which enhances nasal residence time and absorption of drug across nasal-mucosal membrane.

For oral or buccal mucosal administration, a transmucosal product can be formulated with GM1 to be administered via the oral/buccal route using mucoadhesive, quick dissolve tablets or an oral spray formulation. Potential mucoadhesive polymers include hydrophilic polymers containing carboxylic groups such as carbomers (which exhibit the favorable mucoadhesive properties), poly vinyl-pyrrolidone (“PVP”), methyl cellulose (“MC”), sodium carboxy methylcellulose (“SCMC”), hydroxy propyl cellulose (“HPC”), and other cellulose derivatives.

Hydrogels that may also be used include carbopol, polyacrylates and their crosslinked modifications, chitosan and its derivatives, Eudragit-NE30D etc. PEGylating various polymers could also enhance mucoadhesion. Carbopol-934, hydroxypropylmethylcellulose, hydroxyethylcellulose, and sodium carboxymethylcellulose may also be used in various combination ratios, together with a therapeutic amount of GM1 for buccal drug delivery.

For nasal delivery of GM1, devices adapted to provide a means for trans-mucosal delivery of GM1 are inhaler devices for nasal administration of pharmaceutical agents. Such devices as are known to those include nasal inhalers produced by companies such as 3M (______) and others known to those in the art. Typical nasal inhaler devices as are described in U.S. Pat. No. ______, and U.S. Pat. No. ______ each herein incorporated by reference in its entirety. For nasal administration, in a preferred embodiment, the ______ formulation should be deposited near ______ epithelium in order to optimize transport of the brain.

In summary, there currently is no effective neuroprotective or neurorestorative treatment for Parkinson's disease. This new approach, using parenteral transmucosal (e.g., nasal or buccal) delivery of ganglioside therapy enhances functioning of residual dopamine neurons and promotes protection of cognitive and motor functioning, resulting in an easy to use therapy with favorable long-term outcome for patients. GM1 therapy currently needs to be administered by subcutaneous administration or needs to be applied directly to the brain via intraventricular infusion. Neither of these routes of administration is suitable for chronic use in patients with Parkinson's disease. This invention allows GM1 therapy to be administered by routes that will be accessible to any patient and will enhance patient compliance and success of the therapy.

While this description is made with reference to exemplary embodiments, it will be understood by those skilled in the art that various changes may be made and equivalents may be substituted for elements thereof without departing from the scope. In addition, many modifications may be made to adapt a particular situation or material to the teachings hereof without departing from the essential scope. Also, in the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

1. A pharmaceutical composition for treatment or prevention of a central nervous system (CNS) disease or condition in a human patient amenable to treatment by therapeutic administration of an GM1, comprising a formulation for transmucosal administration comprising: GM1 and at least one permeation-enhancing agent effective to enhance transmucosal drug uptake; at least one buffer; at least one solvent; and at least one osmolarity agent.

2. The pharmaceutical composition of claim 1, wherein said CNS disease or condition is Parkinson's disease.

3. The pharmaceutical composition of claim 1, wherein said formulation is an aqueous liquid solution or gel.

4. The pharmaceutical composition of claim 1, wherein said solution is a solution in a liquid.

5. The pharmaceutical composition of claim 1, wherein said permeation-enhancing agent is selected from the group consisting of: alkyl glycosides, tetra-decyl maltoside (TDM), lysophosphatidylcholine, sodium glycochoate, didecanoylphosphatidylcholine (DDPC), cyclodextrins, lauroylcarnitine chloride (LLC), aminated gelatin, SLS and any combination thereof.

6. The pharmaceutical composition of claim 1, wherein said GM1 is either naturally or synthetically derived.

7. The pharmaceutical composition of claim 1, wherein said solvent is water.

8. The pharmaceutical composition of claim 1, wherein said osmolarity agent is selected from the group consisting of sodium chloride, dextrose or sorbitol.

9. The pharmaceutical composition of claim 1, further comprising a co-solvent.

10. The pharmaceutical composition of claim 10, wherein said co-solvent is selected from the group selected from: propylene glycol, polyethylene glycol, ethanol and any combination thereof.

11. The pharmaceutical composition of claim 1, further comprising a viscosity agent, wherein said viscosity agent is a polymer.

12. The pharmaceutical composition of claim 1, wherein said viscosity agent is selected from the group consisting of MC, HPMC, PVP, HEC, NaCMC, microcrystalline cellulose, Hydroxypropyl Cellulose, hydroxyethyl cellulose, polyvinylpyrrolidone and any combination thereof.

13. The pharmaceutical composition of claim 1, further comprising a chelating agent.

14. The pharmaceutical composition of claim 1, wherein said chelating agent is sodium EDTA or disodium EDTA dehydrate.

15. (canceled)

16. (canceled)

17. The pharmaceutical composition of claim 1, wherein said pharmaceutical composition is substantially free of BSE contaminants.

18. (canceled)

19. (canceled)

20. (canceled)

21. (canceled)

22. (canceled)

23. The pharmaceutical composition of claim 1, wherein said permeability enhancing agent is a mucoadhesive agent, wherein nasal resident time and nasal absorption is increased and wherein retention time of the composition and bioavailability of GM1 is enhanced.

24. The pharmaceutical composition of claim 23, wherein said mucoadhesive is a chitosan, a chitosan derivative or a mucoadhesive polymer.

25. The pharmaceutical composition of claim 1, wherein said permeability enhancing agent and comprises a tri-block co-polymer wherein nasal resident time and nasal absorption is increased wherein retention time of the composition and bioavailability of GM1 is enhanced.

26. The pharmaceutical composition of claim 3, wherein said permeability enhancing agent is a mucoadhesive agent, wherein oral/buccal resident time and buccal absorption is increased enhancing retention time of the composition and bioavailability of GM1.

27. The pharmaceutical composition of claim 1, wherein said transmucosal administration involves delivery of said composition to one or both nasal mucosal surfaces of said patient.

28. (canceled)

29. (canceled)

30. A method for treating or preventing a neuromuscular disease or condition in a human patient in need of treatment by therapeutic administration of GM1 comprising the step of administering intranasally to said patient a pharmaceutical composition of claim 1.

31. The method of claim 30, wherein said pharmaceutical composition is administered as a single solution in a multidose or single dose nasal dispenser.

32. (canceled)

33. (canceled)

34. The method of claim 30, wherein said pharmaceutical composition following mucosal administration to said patient yields a peak concentration of said GM1 in a central nervous system tissue or fluid of said subject that is greater than a therapeutic concentration of said GM1 in the plasma of said patient.

35. (canceled)

36. An article of manufacture, comprising: a means for administering a nasal dose and the composition of claim 1.

37. The article of manufacture of claim 61, wherein said means for administering a nasal dose is a nasal dispenser, tampon, sponge, insufflator, nebulizer or pump.

38. (canceled)
